(19) United States
(12) Patent Application Publication
(10) Pub. No.: US 2024/0006037 A1
(43) Pub. Date: Jan. 4, 2024

(54) SYSTEM AND METHOD FOR SIMULTANEOUS GENERATION OF ELECTRONIC MEDICAL RECORDS FOR ARTIFICIAL INTELLIGENCE-ENABLED DYNAMIC IMAGE RECOGNITION

(71) Applicant: SEALONEPLUS TECHNOLOGY LTD., Zhubei City (TW)

(72) Inventor: Tien Hsiu TSO, Zhubei City (TW)

(21) Appl. No.: 17/964,427

(22) Filed: Oct. 12, 2022

(30) Foreign Application Priority Data
Jul. 4, 2022 (TW) 111125019

(51) Int. Cl. G16H 10/60

(52) U.S. Cl. CPC G16H 10/60

(57) ABSTRACT

The present invention discloses a system and method for simultaneous generation of electronic medical records for artificial intelligence-enabled dynamic image recognition. The system performs object-specific recognition of motion pictures generated by medical devices during medical clinical examinations such as ultrasound and endoscopy, and automatically generates locating and marking information for the identified objects. The examiner can control the system with voice commands to confirm the correct object locating and marking information. The system can automatically generate the corresponding standard output format simultaneously. At the end of the clinical examination, the system compiles the relevant data and automatically and simultaneously generates the complete format of the electronic medical record for the examination. The electronic medical record has an interface for editing and confirmation of completion, so that the examiner can make further changes or directly confirm the completion of the medical record.

### BACKGROUND OF THE INVENTION

### 1. Field of the Invention

The present invention relates to a system and method for generation of electronic medical records, especially relates to a system and method for simultaneous generation of electronic medical records for artificial intelligence-enabled dynamic image recognition using artificial intelligence (AI) trained models to recognize objects in motion pictures in real time during medical examinations as a simultaneous aid to physician decision making and automatic report generation.

### 2. Description of the Prior Art

In common medical dynamic imaging examinations, such as endoscopy or ultrasound, the examination result usually produced a graphically integrated reference report, aka an electronic medical record. After the examination is completed, the examiner uses computer equipment and software to complete the electronic medical record. With the advancement of artificial intelligence in image recognition, systems have been developed that use artificial intelligence models to successfully recognize static medical images, such as X-rays, computed tomography (CT), and magnetic resonance imaging (MRI), and to select possible symptoms and levels of disease to assist the examiner or report interpreter in identifying problems. However, with current technology, the examiner or interpreter still needs to complete the report and electronic medical record by manually transcribing the images that have been archived.

### SUMMARY OF THE INVENTION

In view of the deficiencies of the prior-art techniques, the object of the present invention is to provide a system and method for simultaneous generation of electronic medical records for artificial intelligence-enabled dynamic image recognition while medical examination is in progress, in order to maximize the assistance to the examiner or the report interpreter and to automatically produce electronic medical records.

According the one object of the present invention, provides a system for simultaneous generation of electronic medical records for artificial intelligence-enabled dynamic image recognition, comprising: an image receiving and processing module for receiving motion pictures; an object recognition module of Artificial intelligence for motion pictures containing a trained model for recognizing the motion pictures; an object locating and marking module for generating locating and marking information on the recognized motion pictures; a data aggregation module for aggregating the motion pictures with the locating and marking information into a standard output format; and an electronic medical record generator module for outputting the standard output format as an electronic medical record.

According to one aspect of the present invention, the system further comprises a control module for a user to confirm the correctness of the generated locating and marking.

According to one aspect of the present invention, the control module can be controlled by voice commands.

According to one aspect of the present invention, the standard output format is a pre-defined format.

According to one aspect of the present invention, the motion pictures are medical examination images.

According another object of the present invention, provides a method for simultaneous generation of electronic medical records for artificial intelligence-enabled dynamic image recognition, comprising steps of: receiving motion pictures; recognizing the motion pictures with a trained model; generating locating and marking information on the recognized motion pictures; aggregating the motion pictures with the locating and marking information into a standard output format; and outputting the standard output format as an electronic medical record.

According to another aspect of the present invention, the method further comprises step for a user to confirm the correctness of the generated locating and marking information.

According to another aspect of the present invention, the user can use voice commands to confirm the correctness.

According to another aspect of the present invention, the standard output format is a pre-defined format

According to another aspect of the present invention, the motion pictures medical examination images.

In view of the above, the present invention may have one or more of the following advantages:
1. The present invention can automatically interpret motion pictures with artificial intelligence while the medical examination is in progress, helping the examiner or the report interpreter to analyze and identify the contents of the images simultaneously, avoiding missing symptoms and speeding up the examination process.
2. The present invention can automatically generate electronic medical records to assist the examiner or the report interpreter after the examination, saving the examiner's or the report interpreter's time in report writing and improving the quality of the report.

### BRIEF DESCRIPTION OF THE DRAWINGS

In the following, the invention will be described in greater detail by means of preferred embodiments and with reference to the attached drawings, in which

FIG. 1 illustrates a diagram of the system for simultaneous generation of electronic medical records for artificial intelligence-enabled dynamic image recognition.

FIG. 2 illustrates a method flow chart of the method for simultaneous generation of electronic medical records for artificial intelligence-enabled dynamic image recognition.

### DETAILED DESCRIPTION OF THE PREFERRED EMBODIMENT

The present invention will now be described in detail on the basis of exemplary embodiments.

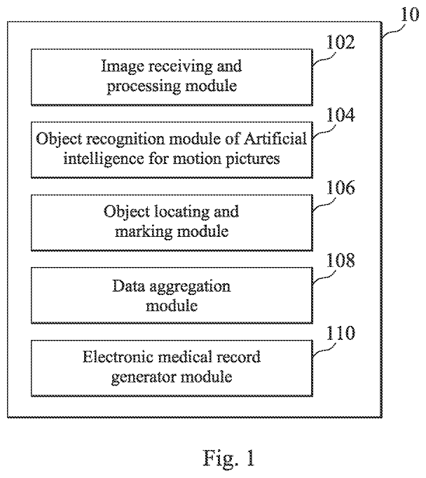

FIG. 1 illustrates a diagram of the system for simultaneous generation of electronic medical records for artificial intelligence-enabled dynamic image recognition. As shown in FIG. 1, the system 10 for simultaneous generation of electronic medical records for artificial intelligence-enabled dynamic image recognition, comprises: an image receiving and processing module 102 for receiving motion pictures; an object recognition module of Artificial intelligence for motion pictures 104 containing a trained model for recognizing the motion pictures; an object locating and marking module 106 for generating locating and marking information on the recognized motion pictures; a data aggregation module 108 for aggregating the motion pictures with the locating and marking information into a standard output format; and an electronic medical record generator module 110 for outputting the standard output format as an electronic medical record.

The system 10 can further comprise a control module (not shown) for a user to confirm the correctness of the generated locating and marking. Also, the control module can be controlled by voice commands of the user.

In the system 10, the standard output format is a pre-defined format, which is the medical standard electronic medical record format.

In the system 10, the motion pictures are medical examination images, listed but not limited to ultrasound and endoscopy images.

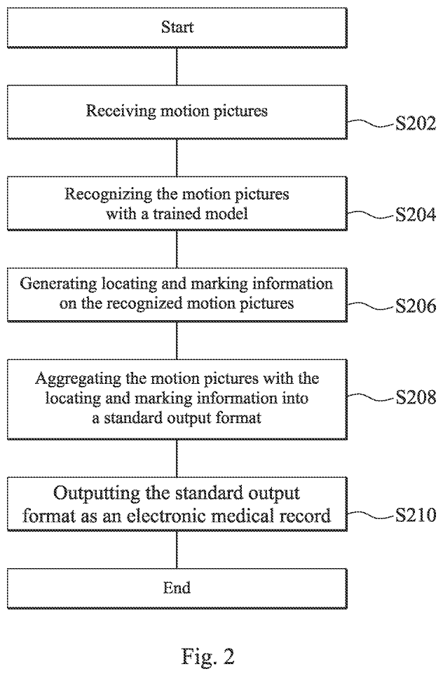

FIG. 2 illustrates a method flow chart of the method for simultaneous generation of electronic medical records tier artificial intelligence-enabled dynamic image recognition. As shown in FIG. 2, the method comprises steps of: receiving motion pictures (S202); recognizing the motion pictures with a trained model (S204); generating locating and marking information on the recognized motion pictures (S206); aggregating the motion pictures with the locating and marking information into a standard output format (S208); and outputting the standard output format as an electronic medical record (S210).

In the step of the above method, the method can further comprise step for a user to confirm the correctness of the generated locating and marking information by using voice commands.

In the step of the above method, the standard output format is a pre-defined format, which is the medical standard electronic medical record format.

In the step of the above method, the motion pictures are medical examination images, listed but not limited to ultrasound and endoscopy images.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A system for simultaneous generation of electronic medical records for artificial intelligence-enabled dynamic image recognition, comprising:
an image receiving and processing module for receiving motion pictures;
an object recognition module of Artificial intelligence fur motion pictures containing a trained model for recognizing the motion pictures;
an object locating and marking module for generating locating and marking information on the recognized motion pictures;
a data aggregation module for aggregating the motion pictures with the locating and marking information into a standard output format; and
an electronic medical record generator module for outputting the standard output format as an electronic medical record.

2. The system according to claim 1, wherein the system further comprises a control module for a user to confirm the correctness of the generated locating and marking.

3. The system according to claim 2, wherein the control module can be controlled by voice commands.

4. The system according to claim 1, wherein the standard output format is a pre-defined format.

5. The system according to claim 1, wherein the motion pictures are medical examination images.

6. A method for simultaneous generation of electronic medical records for artificial intelligence-enabled dynamic image recognition, comprising steps of:
receiving motion pictures;
recognizing the motion pictures with a trained model;
generating locating and marking information on the recognized motion pictures;
aggregating the motion pictures with the locating and marking information into a standard output format; and
outputting the standard output format as an electronic medical record.

7. The method according to claim 6, wherein the method further comprises step for
a user to confirm the correctness of the generated locating and marking information.

8. The method according to claim 7, wherein the user can use voice commands to confirm the correctness.

9. The method according to claim 6, wherein the standard output format is a pre-defined format

10. The method according to claim 6, wherein the motion pictures are medical examination images.
